(19) United States
(12) Patent Application Publication
(10) Pub. No.: US 2016/0345896 A1
(43) Pub. Date: Dec. 1, 2016

(54) SYSTEM FOR IDENTIFYING TISSUE CHARACTERISTICS OR PROPERTIES UTILIZING RADIOMETRIC SENSING

(71) Applicant: MERIDIAN MEDICAL SYSTEMS, LLC, Portland, ME (US)

(72) Inventor: Robert Allison, Rancho Palos Verdes, CA (US)

(21) Appl. No.: 15/117,115

(22) PCT Filed: Feb. 6, 2015
(86) PCT No.: PCT/US15/14827

§ 371 Date: Aug. 5, 2016

Related U.S. Application Data
(60) Provisional application No. 61/936,576, filed on Feb. 6, 2014.

(51) Int. Cl. A61B 5/00; A61B 5/05; A61B 18/14; A61B 18/02; A61B 18/18; A61B 18/20; A61B 5/01; A61B 5/053

(52) U.S. Cl. CPC A61B 5/4836; A61B 5/01; A61B 5/0507; A61B 5/053; A61B 18/02; A61B 18/1815; A61B 18/20; A61B 18/14; A61B 2018/00577

(57) ABSTRACT

The invention provides, inter alia, devices and associated methods to measure tissue state of a biological tissue, e.g., using radiometric sensing to, e.g., simultaneously measure temperature and tissue state. These devices and methods can be used in the course of ablation therapy, for example.

### BACKGROUND OF THE INVENTION

This application claims the benefit of U.S. Provisional Application No. 61/936,576, filed on Feb. 6, 2014.

The entire teachings of the above application(s) are incorporated herein by reference.

There is broad clinical utility to recognizing characteristics of tissue in the human body, ranging from diagnosis of disease by recognizing a state of target tissue or anomalies in a target tissue to recognizing change in tissue characteristics during or following therapy. As an example, feedback given to a treating clinician during application of energy (i.e., ablative techniques) can provide vital information as to the result of that intervention, ultimately resulting in more consistent, safer therapies and improved patient outcomes. Ablative techniques involving a range of energy platforms are employed in a myriad of treatments including, but not limited to, cardiac ablation, denervation of sympathetic nerves in the renal arteries, carotid body modulation, ablation of soft tissue tumors, prostate ablation and ablative therapies to the esophagus. These and any number of other ablative interventions benefit from feedback as to the effect of the delivery of energy to a target tissue. Lacking valuable feedback results in both inadequate and inconsistent therapies, as well as over-treatment with unintended clinical consequences.

Accordingly, a need exists for devices, and associated methods, that can provide useful feedback on a tissue, such as the state of a tissue (e.g., disease state) by simultaneously measuring tissue temperature and state, for example, during ablation therapy.

### SUMMARY OF THE INVENTION

The invention provides, inter alia, devices and associated methods to measure tissue state of a biological tissue, e.g., using radiometric sensing to, e.g., simultaneously measure temperature and tissue state. These devices and methods can be used in the course of, inter alia, ablation therapy.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a schematic diagram of one exemplary embodiment provided by the invention. In this particular exemplification, tissue impedance is measured passively (e.g., by measuring the magnitude of tissue impedance mismatch).

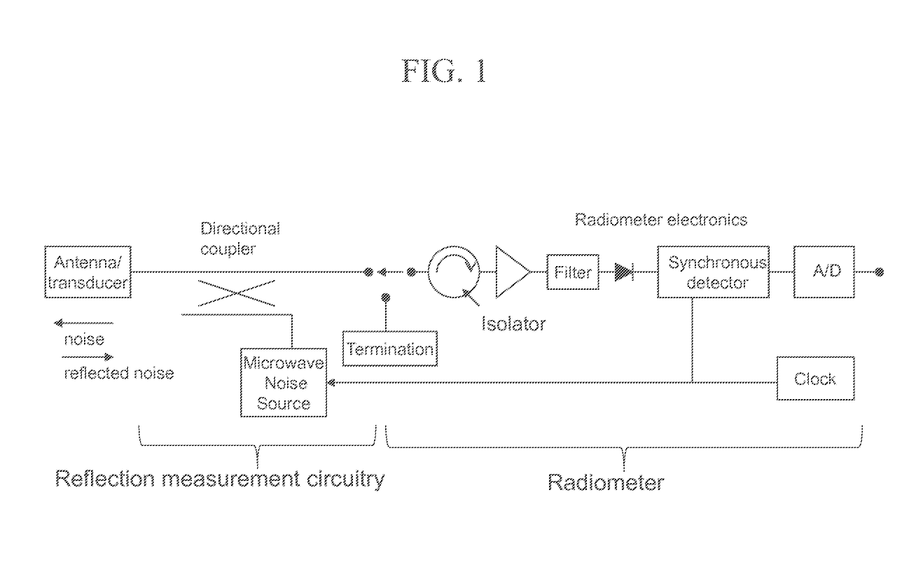

FIG. 2 is a schematic diagram of another exemplary embodiment provided by the invention. In this exemplification, tissue impedance is measured actively (e.g., by measuring the magnitude and phase of tissue impedance mismatch). The boxed elements are readily adaptable to an integrated circuit, e.g., MMIC.

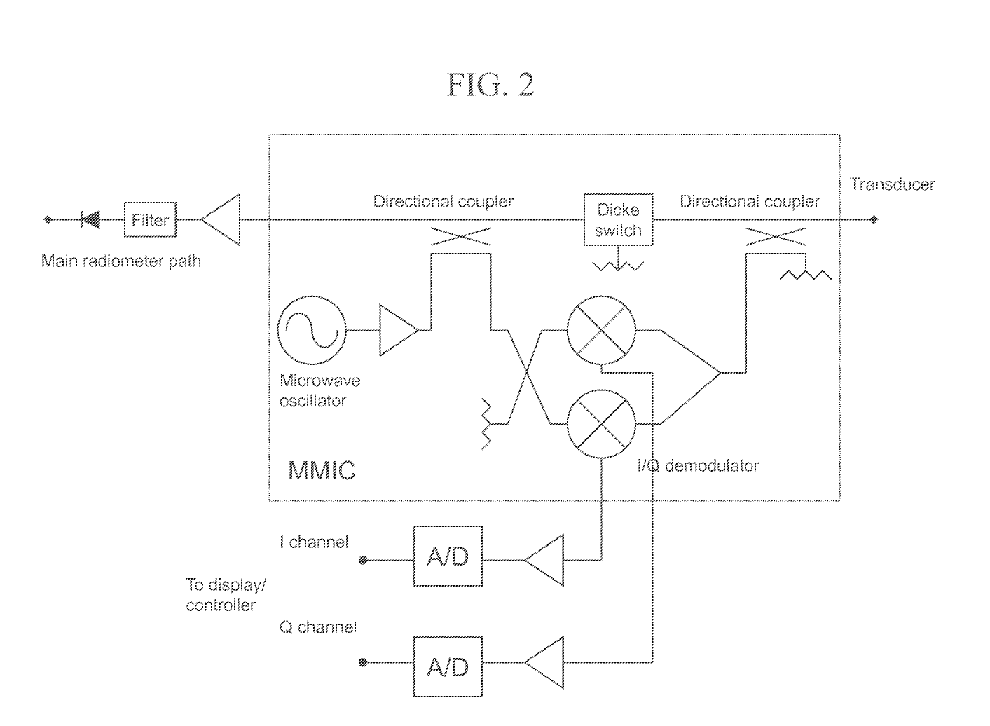

FIG. 3 is a schematic diagram of an exemplary I/Q detector.

FIG. 4 is a schematic diagram of an exemplary detector block.

FIG. 5 is a schematic diagram of an exemplary test setup.

FIG. 6 is a plot of detected output versus load phase shifter setting.

FIG. 7 is a plot of measured phase versus load phase.

FIG. 8 is a plot of C band transducer measurements.

### DETAILED DESCRIPTION OF THE INVENTION

A description of example embodiments of the invention follows.

The invention provides devices and associated methods of using them to, for example, measure tissue state by measuring tissue impedance. In certain embodiments, the devices can simultaneously measure the temperature and impedance of a tissue, e.g., during an ablation procedure. Accordingly, in one aspect, the invention provides devices for simultaneously measuring temperature and impedance of a biological tissue, comprising a radiometer configured to measure temperature, an antenna coupled to the radiometer, and circuitry configured to measure tissue impedance.

Any suitable radiometer can be used consonant with the invention. In particular embodiments, the radiometer is a Dicke radiometer. Exemplary radiometers are also described in U.S. Pat. Nos. 6,496,738 and 6,210,367, which are incorporated by reference in their entirety. The devices (such as radiometers) described herein and in the above patents can be configured on a single integrated circuit. Such integrated circuits can further comprise elements described in the devices provided by the invention.

Microwave frequencies are typically used for detection of temperature and impedance in the present invention, including microwave energy from about 1 to about 30 GHz, e.g., about 1-5 GHz, more particularly, about 3-5 GHz, and, still more particularly, about 4 GHz, e.g., 4 GHz +/−200 MHz. Suitable frequencies can be selected by the skilled artisan based on well-understood principles, e.g., the scanning depth in a target sample, e.g., a tissue, and associated parameters, such as antenna size relative to the application (e.g., for use in an adult versus a neonate, or for internal (more limited antenna size) versus external use (larger antennas are more easily used)) on a body.

Any antenna suitable for use with the radiometer element of a device provided by the invention can be used and configured for the appropriate application. In certain embodiments, the antenna may be a disposable or an easily replaceable element.

In some embodiments, the circuitry configured to measure tissue impedance is configured to measure the magnitude of tissue impedance mismatch.

As used herein “tissue impedance mismatch” means the deviations between impedance measured by the radiometer and the antenna. Impedance can be measured by any means and refers to measurements of the relative permittivity (dielectric constant) and can include discreet or continuous measurements of magnitude (relative or absolute) and/or phase, as well as rates of change (e.g., first, second, or higher-order derivatives) or various transformations thereof. Exemplary transformations include time and/or space integrals or normalization (such as mean variance normalization) of any of these measurements (or transforms) to predetermined standards. Such predetermined standards can, e.g., be stored in a database, which may optionally be stored in a nontransient computer-readable medium in a device provided by the invention or in an external database communicatively coupled to a device provided by the invention. Impedance can be measured at a single frequency or over a bandwidth (e.g., at about 2, 3, 4, 5, 6, 7, 8, 9, 10, 20, 50, 100, or more frequencies).

In certain embodiments, the circuitry configured to measure tissue impedance is configured to passively measure the magnitude of tissue impedance mismatch. In more particular embodiments, the circuitry configured to measure tissue impedance comprises a directional coupler and a microwave noise source.

In other embodiments, the circuitry configured to measure tissue impedance is configured to actively measure the magnitude of tissue impedance mismatch. In certain embodiments, the circuitry configured to measure tissue impedance further measures the phase of the tissue impedance mismatch. In more particular embodiments, the circuitry configured to measure tissue impedance comprises a quadrature detection scheme. In certain particular embodiments, the circuitry configured to measure tissue impedance comprises a directional coupler, a microwave oscillator, and an I/Q demodulator. In some particular embodiments, the circuitry configured to measure tissue impedance comprises an amplitude modulator, such as a Dicke switch. In certain particular embodiments, the circuitry configured to measure tissue impedance is configured to measure the complex tissue impedance.

In any of the aspects or embodiments of the invention, the radiometer and circuitry configured to measure tissue impedance can be formed on a single integrated circuit.

In certain embodiments, the devices provided by the invention are suitable for use as catheter-based devices. Catheter-based devices include, but are not limited to, devices suitable for internal applications in the human body, and, in particular embodiments, minimally-invasive techniques, e.g., procedures that are not fully open. Exemplary catheter-based applications include cardiac ablation, denervation of sympathetic nerves in the renal arteries, carotid body modulation, ablation of soft tissue tumors, prostate ablation and ablative therapies to the esophagus.

In other particular embodiments, a device provided by the invention is a remote device, e.g., one untethered when in use.

In certain embodiments, a device provided by the invention is a handheld device or incorporated into a handheld device.

In particular embodiments, a device provided by the invention comprises a closed power source, such as a battery.

In some embodiments of any of the aspects and embodiments of the invention, the device comprises a non-transient storage medium for recording the measured temperature and impedance.

In certain embodiments of any of the aspects and embodiments of the invention, the device further comprises a non-transient storage medium with reference values of tissue impedance stored thereon. For example, in some embodiments, the reference values are suitable for identifying or classifying tissues selected from adipose tissue, muscle (smooth, skeletal, and cardiac), vasculature (arteries and veins), esophagus, thyroid cartilage, bone, lung, kidney, liver, spleen, pancreas, stomach, blood, intestine (large or small), appendix, brain, spinal cord, bladder, uterus, breast, heart, skin, or prostate, including any of the foregoing in a healthy, compressed (e.g., as a measure of device depth in the tissue), or diseased (e.g., plaqued/occluded (e.g., atherosclerotic), burnt, scarred, cirrhotic, or cancerous) state. That is, the reference values are compared to measured values by any means, such as through use of a processor integrated in a device provided by the invention, or a separate processor in communication—either by direct physical linkage or radion/wireless communication—with a device provided by the invention. The processor may be communicatively coupled to an embodiment of the invention through any communication means known in the art. These processors can optionally make a classification of the measured sample as one of the above mentioned tissues by using the reference values in the storage medium. Additional methods and devices for detecting vulnerable plaques, e.g., based on thermal properties measured by the radiometer, can be incorporated into the devices and methods provided by the invention and are described in U.S. Pat. No. 6,932,776, which is incorporated by reference in its entirety.

In certain embodiments, the device is coupled to a user-readable display. Coupling may be done through any means known in the art and may encompass both physical coupling, e.g., through a wire, as well as radio/wireless communication.

In any of the preceding aspects and embodiments, the device can further comprise a means for tissue ablation. In particular embodiments, the tissue ablation can be by microwave, radiofrequency, ultrasound, laser, or cryogenic ablation. In more particular embodiments, the means for tissue ablation is microwave or radiofrequency ablation.

In another aspect, the invention provides methods of simultaneously measuring the temperature and impedance of a biological tissue. These methods entail placing the tissue in close proximity with the device of any one of the aspects or embodiments described herein. In certain embodiments, close proximity is less than about 2 cm, e.g., less than about 19, 18, 17, 16, 15, 14, 13, 12, 11, 10, 9, 8, 7, 6, 5 mm, or less, e.g., about: 5-1 mm. In particular embodiments, the device is in physical contact with the biological tissue.

In certain embodiments, the tissue is selected from adipose tissue, muscle (smooth, skeletal, and cardiac), vasculature (arteries and veins), connective tissue, esophagus, thyroid cartilage, bone, lung, kidney, liver, spleen, pancreas, stomach, blood, intestine (large or small), appendix, brain, spinal cord, bladder, uterus, breast, heart, skin, or prostate, including any of the foregoing in healthy or diseased (e.g., atherosclerotic, burnt, scarred, cirrhotic, or cancerous) states.

In particular embodiments, the tissue is in a mammalian subject. In more particular embodiments, the mammalian subject is a human.

In certain particular embodiments, the methods further comprise detecting whether the device is in physical contact with the biological tissue, and, optionally, the depth of the device in the tissue, which may be determined based on a change in impedance.

In particular embodiments, the methods comprise detecting a disease state of the biological tissue based on a change in impedance.

In certain particular embodiments, the biological tissue is undergoing active ablation.

In another aspect, the invention provides a handheld device for actively measuring tissue impedance in a biological tissue of a human body. Such a device may comprise an antenna, a directional coupler, a microwave oscillator, and an I/Q demodulator.

According to yet another aspect, the invention provides a handheld device for passively measuring tissue impedance in a biological tissue of a human body, comprising an antenna, a directional coupler, and a microwave noise source.

In either of the preceding two aspects, the device may, in some embodiments, further comprise a radiometer configured to measure temperature—i.e., the device can be configured according to any aspect or embodiment described herein.

In a related aspect, the invention also provides an article of manufacture comprising the device of any one of the aspects or embodiments described herein and, optionally, instructions for use.

### EXEMPLIFICATION

This example describes an I/Q Extravasation Detector.

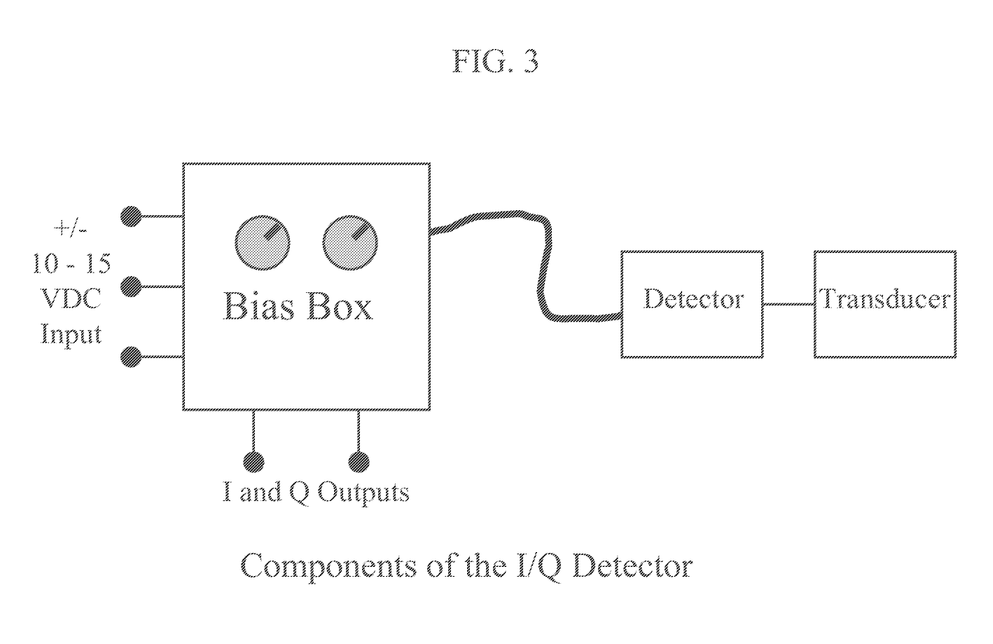

The I/Q detector consisted of three parts as shown in FIG. 3: detector, transducer, and bias box. There were two potentiometers to adjust the mixer bias offset. The I/Q detector outputs were between +/−300 mv. A block diagram of the detector system is shown in FIG. 3.

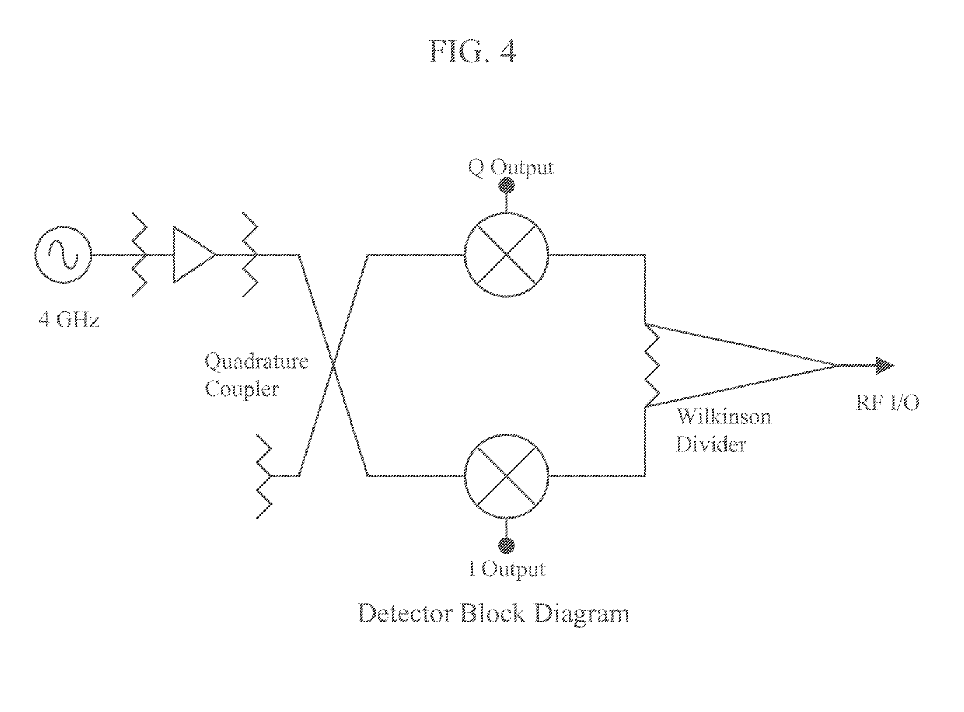

A block diagram of the detector circuit is shown in FIG. 4. It consisted of an oscillator, a buffer amplifier, a quadrature coupler, two single balanced mixers, and a Wilkinson power divider (combiner). In this unit the oscillator produced 4.04 GHz. The buffer amplifier was intended to minimize load pulling. The single balanced mixers intentionally had poor isolation so that about −5 dBm was transmitted to the transducer. The mixer diodes were biased so that they could be run at low LO power and have control of the sensitivity. A Wilkinson divider combined the outputs of the two mixers and split the returning signal with equal phase.

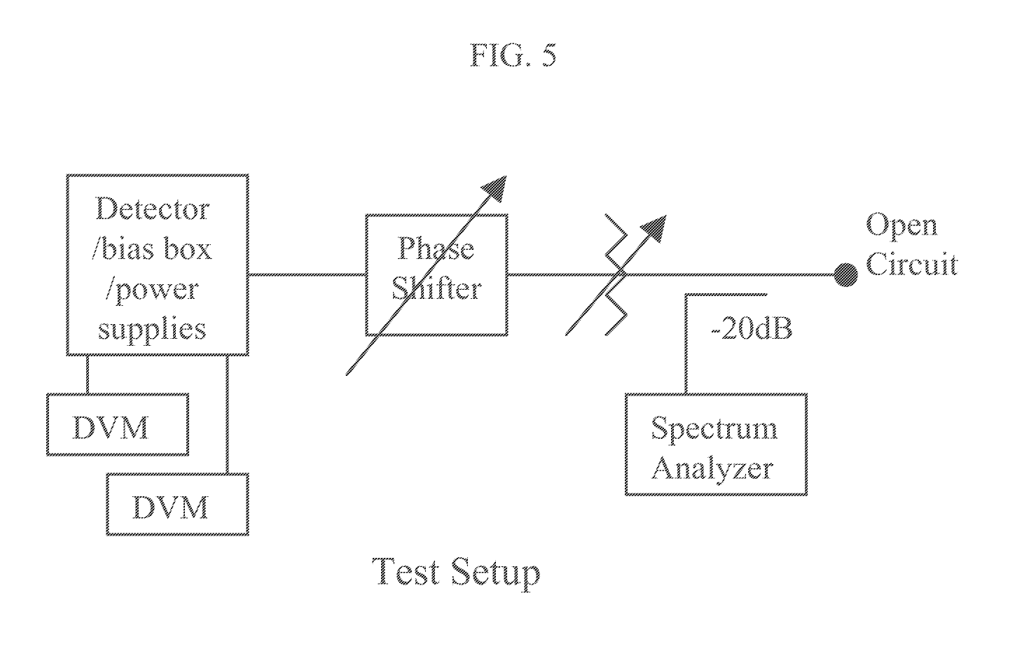

The following discussion of detector measurements references the device with the transducer removed and coaxial loads attached. The test setup is shown in FIG. 5. An open circuit at the end of a cascade of coaxial variable phase shifter and variable attenuator comprised an adjustable load. A coupler allowed the measurement of power and frequency with a spectrum analyzer.

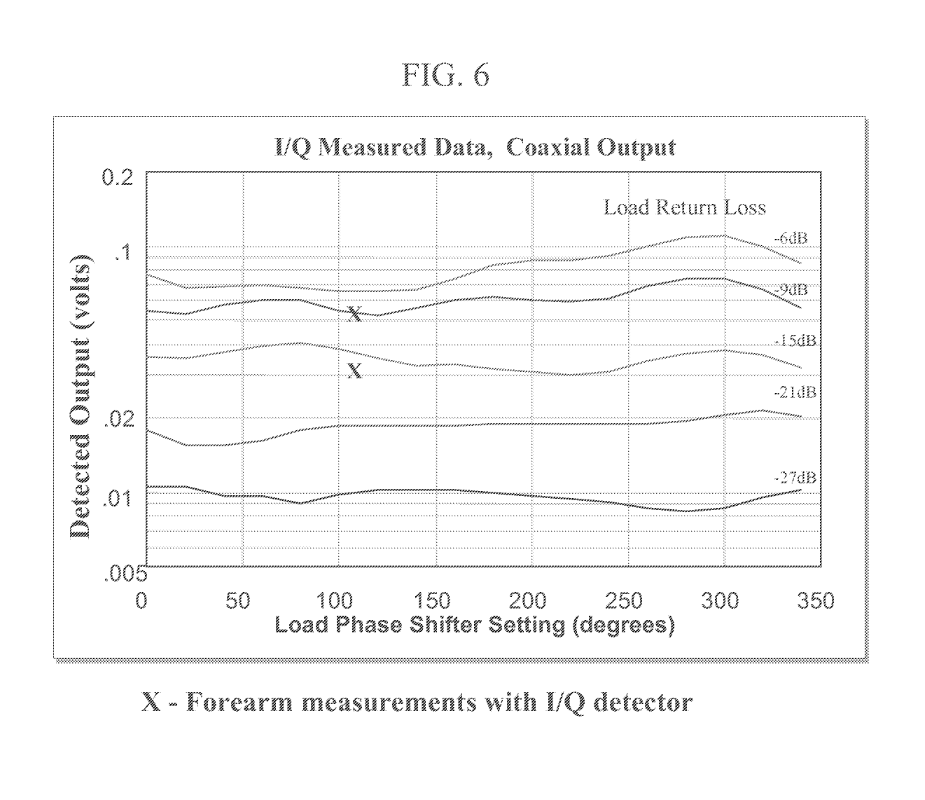

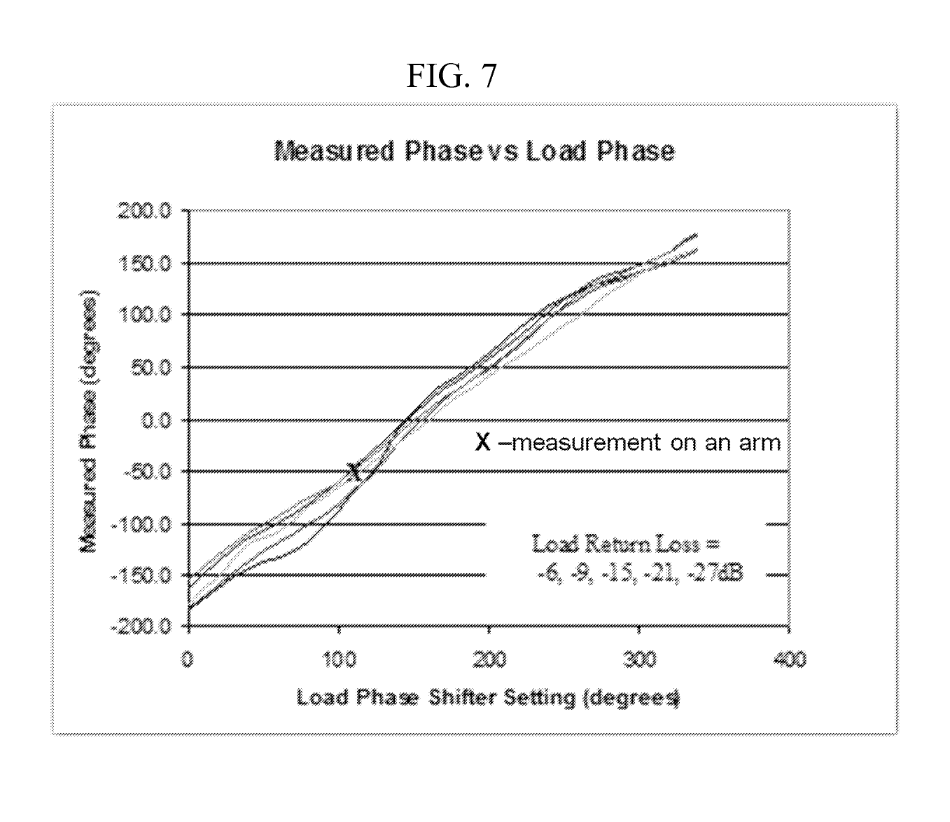

I and Q output voltages were recorded for several load reflection coefficient magnitudes in 20 degree phase increments covering 360 degrees. The output plots of FIGS. 6-8 were calculated from this data. The equations used were

Detected output (volts)={(Iout−Ioffset)²+(Qout−Qoffset)²}^{1/2}

Detected Phase=Tan⁻¹{(Qout−Qoffset)/(Iout−Ioffset)}

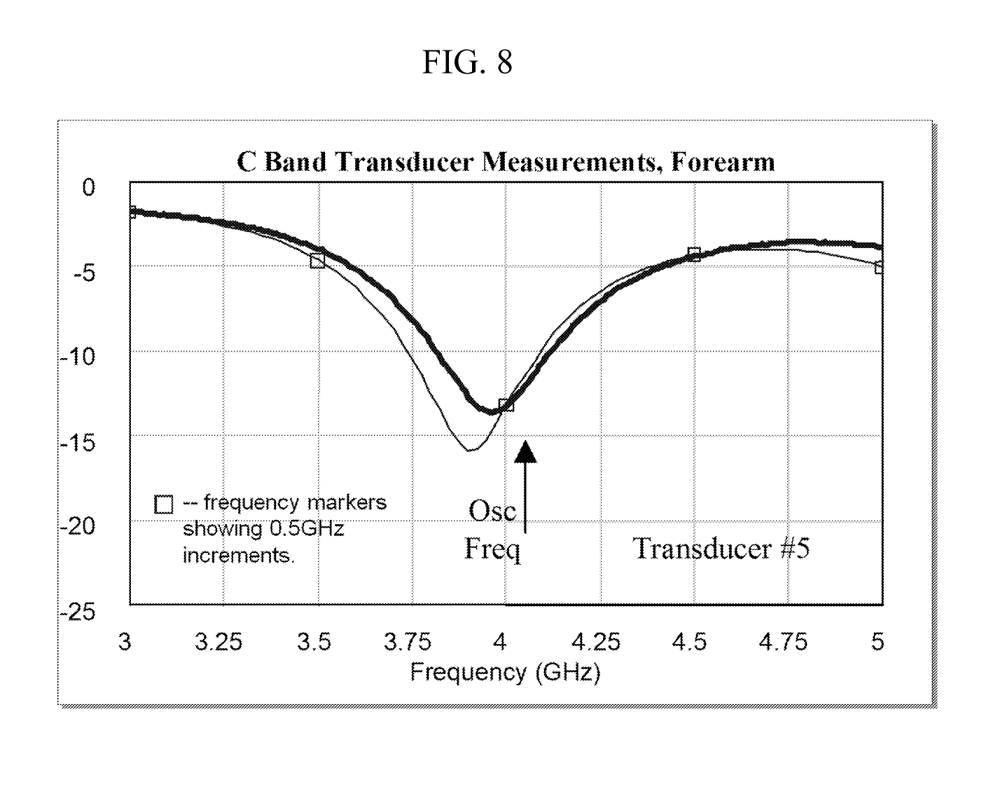

The detector gave a reasonably sensitive and unambiguous response for a load return loss range of greater than 20 dB at all reflection phase angles. The transducer was attached and two measurements of a subject's forearm were made and are displayed on these plots. For comparison, two network analyzer measurements of the transducer and the subject forearm are shown in FIG. 8. The I/Q detector measurements of about 16 dB and 9 dB were somewhat consistent with the network analyzer measurement.

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

Headings used in this application are for convenience only and do not affect the interpretation of this application.

Preferred features of each of the aspects provided by the invention are applicable to all of the other aspects of the invention mutatis mutandis and, without limitation, are exemplified by the dependent claims and also encompass combinations and permutations of individual features (e.g., elements, including numerical ranges and exemplary embodiments) of particular embodiments and aspects of the invention, including the working examples. For example, particular experimental parameters exemplified in the working examples can be adapted for use in the claimed invention piecemeal without departing from the invention. For example, for materials that are disclosed, while specific reference of each of the various individual and collective combinations and permutations of these compounds may not be explicitly disclosed, each is specifically contemplated and described herein. Thus, if a class of elements A, B, and C are disclosed as well as a class of elements D, E, and F and an example of a combination of elements A-D is disclosed, then, even if each is not individually recited, each is individually and collectively contemplated. Thus, in this example, each of the, combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application, including elements of a composition of matter and steps of method of making or using the compositions.

The forgoing aspects of the invention, as recognized by the person having ordinary skill in the art following the teachings of the specification, can be claimed in any combination or permutation to the extent that they are novel and non-obvious over the prior art—thus, to the extent an element is described in one or more references known to the person having ordinary skill in the art, they may be excluded from the claimed invention by, inter alia, a negative proviso or disclaimer of the feature or combination of features.

It should be understood that the example embodiments described above may be implemented in many different ways. In some instances, the various methods and machines described herein may be implemented by a physical, virtual, or hybrid general purpose computer, or a computer network environment.

Embodiments or aspects thereof may be implemented in the form of hardware, firmware, or software. If implemented in software, the software may be stored on any non-transient computer-readable medium that is configured to enable a processor to load the software or subsets of instructions thereof. The processor then executes the instructions and is configured to operate or cause an apparatus to operate in a manner as described herein.

Further, firmware, software, routines, or instructions may be described herein as performing certain actions and/or functions of the data processors. However, it should be appreciated that such descriptions contained herein are merely for convenience and that such actions, in fact, result from computer devices, processors, controllers, or other devices executing the firmware, software, routines, instructions, etc.

It should also be understood that the schematics may include more or fewer elements, be arranged differently, or be represented differently. But it should further be understood that certain implementation may dictate that the schematic be implemented in a particular way.

While this invention has been particularly shown and described with references to example embodiments thereof, it will be understood by those skilled in the art that various changes in form and details may be made therein without departing from the scope of the invention encompassed by the appended claims.

1. A device for simultaneously measuring temperature and impedance of a biological tissue, comprising a radiometer configured to measure temperature, an antenna coupled to the radiometer, and circuitry configured to measure tissue impedance.

2. The device of claim 1, wherein the circuitry configured to measure tissue impedance is configured to measure the magnitude of tissue impedance mismatch.

3. The device of claim 2, wherein the circuitry configured to measure tissue impedance is configured to passively measure the magnitude of tissue impedance mismatch.

4. (canceled)

5. The device of claim 2, wherein the circuitry configured to measure tissue impedance is configured to actively measure the magnitude of tissue impedance mismatch.

6. The device of claim 5, wherein the circuitry configured to measure tissue impedance further measures the phase of the tissue impedance mismatch.

7. The device of claim 6, wherein the circuitry configured to measure tissue impedance comprises a quadrature detection scheme.

8. The device of claim 6, wherein the circuitry configured to measure tissue impedance comprises a directional coupler, a microwave oscillator, and an I/Q demodulator.

9. (canceled)

10. (canceled)

11. (canceled)

12. (canceled)

13. (canceled)

14. (canceled)

15. (canceled)

16. (canceled)

17. (canceled)

18. The device of claim 1, wherein the device further comprises a non-transient storage medium with reference values of tissue impedance stored thereon, the reference values suitable for identifying tissues selected from adipose tissue, muscle, vasculature, esophagus, thyroid cartilage, bone, lung, kidney, liver, spleen, pancreas, stomach, blood, intestine, appendix, brain, spinal cord, bladder, uterus, breast, hearts skin, or prostate, including any of the foregoing in healthy, compressed, or diseased state.

19. (canceled)

20. The device of claim 1, wherein the device further comprises a means for tissue ablation.

21. The device of claim 20, wherein the means for tissue ablation is selected from microwave, radiofrequency, ultrasound, laser, or cryogenic ablation.

22. The device of claim 21, wherein the means for tissue ablation is microwave or radiofrequency ablation.

23. A method of simultaneously measuring the temperature and impedance of a biological tissue, comprising placing the tissue in close proximity with a device including a radiometer configured to measure temperature, an antenna coupled to the radiometer, and circuitry configured to measure tissue impedance.

24. The method of claim 23, wherein the tissue is selected from adipose tissue, muscle, vasculature, connective tissue, esophagus. thyroid cartilage, bone, lung, kidney, liver, spleen, pancreas, stomach, blood, intestine, appendix, brain, spinal cord, bladder, uterus, breast, heart, skin, or prostate, including any of the foregoing in healthy or diseased state.

25. The method of claim 24, wherein the tissue is in a mammalian subject.

26. The method of claim 25, wherein the mammalian subject is a human.

27. The method of claim 23, further comprising detecting whether the device is in physical contact with the biological tissue, and, optionally, the depth of the device in the tissue, based on a change in impedance.

28. The method of claim 23, further comprising detecting a disease state of the biological tissue based on a change in impedance.

29. The method of claim 23, wherein the biological tissue is undergoing active ablation.

30. A handheld device for actively measuring tissue impedance in a biological tissue of a human body, comprising an antenna, a directional coupler, a microwave oscillator, and an I/Q demodulator.

31. (canceled)

32. (canceled)

33. (canceled)

34. (canceled)
